(19) United States
(12) Patent Application Publication
(10) Pub. No.: US 2012/0022433 A1
(43) Pub. Date: Jan. 26, 2012

(54) METHODS FOR INCREASING TRICHOGENICITY OF DERMAL CELLS

(75) Inventors: Ying Zheng, West Chester, PA (US); Arben Nace, Landenberg, PA (US); Luke Sergott, Philadelphia, PA (US); Michael Ensslin, Atlanta, GA (US); Ying Homan, Ambler, PA (US); Kurt Stenn, Princeton, NJ (US)

(73) Assignee: Aderans Research Institute, Inc.

(21) Appl. No.: 13/250,536

(22) Filed: Sep. 30, 2011

Related U.S. Application Data
(62) Division of application No. 12/353,699, filed on Jan. 14, 2009.

(51) Int. Cl. A61M 37/00; C12N 5/071

(52) U.S. Cl. 604/20; 604/22; 604/187; 435/325

(57) ABSTRACT

Methods for increasing trichogenic activity of populations of dermal cells by inducing local trauma to skin tissue that serves as a source of the dermal cells are provided. Methods for obtaining dermal cells with increased trichogenic activity and for using the disclosed dermal cells to implant into a mammalian host at a site of desired follicle generation are also provided.

### FIELD OF THE INVENTION

The invention is generally related to cell physiology, in particular to methods for increasing the follicle-forming ability of skin-derived dermal cells.

### BACKGROUND OF THE INVENTION

By 30 years of age, approximately 30% of white males have begun developing alopecia and by 50 years, half of the same population is affected. The typical defined patterns of male pattern hair loss are well known and have been described by the Hamilton scale (Hamilton, Ann. N.Y. Acad. Sci., 53:708-28 (1951)) and later modified in the norwood-Hamilton scale (Norwood, South Med. J., 68(11):1359-65 (1975)). According to estimates from the Unites States Food and Drug Administration (FDA), 40 million men and 20 million women experience inherited hair loss (Hanover, FDA Consumer, 31:3 (1997)).

Therapeutic and cosmetic approaches have been undertaken for androgenetic alopecia. Many, if not most, do not work or are merely temporary or partial solutions, which are expensive and often are not free of possible dangerous or adverse secondary effects. To date only two drugs are approved by the FDA for the treatment of androgenetic alopecia. Minoxidil (Rogaine®) is a vasodilator that claims to stimulate the conversion of vellus hair into terminal hair at the vertex of the scalp (U.S. Pat. No. 4,139,619). A 5% concentration applied as a topical solution is reported to regrow some fine hair in the vertex scalp region of 50% of the users after a year of constant use. As a vasodilator there are safety concerns about possible secondary adverse effects. Finasteride (Propecia®), a 5 α-reductase type 2 inhibitor, prevents the conversion of testosterone into dihydrotestosterone (DHT). This agent, approved in 1997 for the oral treatment of androgenetic alopecia (U.S. Pat. Nos. 5,516,779; 4,377,584; and 4,760,071), has been reported to be effective in reducing further hair loss in 52% of the users after a year of constant use. Women in reproductive years must be careful not to have any contact with the medication because of known risk of birth defects. Recent reports indicate that the use of both compounds (minoxidil topically plus finesteride orally) might slightly increase the percentage of males regrowing some hair after one year of constant use. Several herbal remedies that claim to help alleviate baldness are available over the counter including pygeum, saw palmetto, stinging nettles and green tea.

Another approach available for treating hair loss is surgical transplants. In 2003, it was estimated that 120,000 people in the United States, approximately 90% of them men, underwent hair transplant surgery. Hair transplantation is an outpatient surgical procedure that involves the autologous transplantation of entire hair follicles from unaffected to affected areas. Currently, follicular unit transplantation is the most commonly utilized technique. The principal unmet clinical need of follicle based hair transplantation is the limitation of the donor follicles available for transplantation. Current surgical hair replacement involves excising a 1 cm by 15-30 cm strip of occipital scalp (15-30 cm²). The hair follicles on this strip of skin are isolated, with a typical yield of 1000 to 2500 follicular unit grafts. These follicular grafts are then implanted into the balding area of the subject. Surgical hair replacement can only redistribute existing hair follicles and does not generate new hair growth.

The mammalian hair follicle is a system which depends on extensive and intimate epithelial and mesenchymal interactions. Throughout the lifetime of the individual the mature hair follicle undergoes a cycle of growth, regression and rest followed by hair shaft shedding and then repetitive recycling. The hair follicle undergoes a cycle of hair growth (anagen) followed by regression (catagen), and quiescence (telogen) until a new hair shaft is generated in the existing follicle during the subsequent anagen phase (Hardy, et al., Trends in Genetics, 8:55-61 (1992)). The hair shaft is derived from the epithelial matrix cells at the base of the follicle, but a cluster of dermal cells ensheathed by the matrix cells, known as dermal papilla, is thought to supply inductive signals required for hair outgrowth. It has also been found that the dermal papilla (or follicular papilla) when dissected free of the follicle and implanted under a neutral epithelial layer will induce a new hair follicle (Stenn and Taus, Physiol. Reviews, 81:449-94 (2001), and references therein). It is known that hair shaft plucking or skin surface irritation can induce the hair cycle (Stenn and Paus, Physiol. Reviews, 81:449-94 (2001), and references therein). How hair shaft plucking induces a new anagen growth phase has not been elucidated. Several studies have examined the ability of dermal papilla and epidermal cells at different stages of the hair cycle to induce new hair shaft growth. For example, one study determined that hair-forming frequencies of follicles are affected by the hair cycle stages of both the dermal papilla and the follicular epithelial cells (Iida, et al., Differentiation, 75:371-81 (2007)). The purpose of the hair cycle has been debated but the cycle undoubtedly allows the follicle to change the character and color of its hair shaft, in response to seasonal needs; moreover, by periodically shedding its surface hair the skin surface is periodically cleansed.

While neofolliculogenesis is not generally believed to occur normally in the adult state, new follicle formation can be induced experimentally by cellular manipulation and by extensive trauma (Ito, et al., Nature, 447:316-320 (2007)). It is well known that specific cells within the hair follicle, including epidermal stem cells and dermal papilla cells, have the capacity to induce follicle neogenesis. Cell-based hair transplants, utilizing hair-forming cells dissociated from donor skin and expanded in tissue culture, hold the promise of creating an increase in available donor tissue for transplantation through cell expansion. Attempts have been made to exploit the inductive capabilities of these cells, including injecting dermal papilla cells directly into the skin and implanting plucked hairs carrying epithelial cells having various proliferative and differentiative characteristics. However, there is a need for methods to increase the ability of isolated populations of dermal cells to form new functional hair follicles in the transplant recipient, a process referred to as trichogenesis.

Therefore, it is an object of the invention to provide methods for increasing the trichogenic ability of populations of dermal cells.

It is another object of the invention to provide methods for obtaining populations of dermal cells with increased trichogenic ability.

It is yet another object of the invention to provide methods for inducing new hair follicle growth in a subject using populations of dermal cells with increased trichogenicity.

It is yet another object of the invention to provide methods for treating hair loss in a subject by administering dermal cells with increased trichogenic ability.

### SUMMARY OF THE INVENTION

Methods for increasing trichogenic activity of a population of dermal cells by inducing local trauma to skin tissue that serves as a source of the dermal cells are provided. In one embodiment, the population of dermal cells contains dermal papilla cells. Local trauma to skin tissue that is effective to increase dermal cell trichogenicity can be achieved by a variety of suitable mechanical and chemical means. Increased trichogenicity of dermal cells is achieved after approximately 30 minutes to 4 weeks after the traumatic event. In one embodiment, the effect of the local trauma does not extend to skin beyond the skin that is directly traumatized.

Methods for obtaining dermal cells with increased trichogenic activity are also provided. The methods include the steps of obtaining skin tissue that has been subjected to local trauma after a period of time and dissociating the skin tissue into populations of dermal cells and epidermal cells. The dissociated cells may optionally be cultured and expanded.

Dermal cells with increased trichogencicity obtained using the disclosed methods may be used to implant into a mammalian host at a site of desired follicle generation. The dermal cells are optionally combined with epidermal cells, and optionally, additional cell types prior to introduction into the subject. The cells may be xenogenic, allogenic or autologous, but are preferably autologous.

### BRIEF DESCRIPTION OF THE DRAWINGS

FIG. 1 is a bar graph showing the effect of hair plucking on the trichogenic activity of cells isolated from the dermis after the traumatic plucking event. The trichogenic activity of the dermal cells was measured using the Aderans Hair Patch Assay™ which measures the ability of the dermal cells to form new follicles when implanted with epidermal cells into immunocompromised mice. Data are expressed as the average number of hair follicles as a function of time after the plucking event and the error bars represent standard deviation.

FIG. 2 is a bar graph comparing the trichogenicity of dermal cells isolated from skin from which the hair was plucked to that of dermal cells isolated from areas of skin immediately adjacent in which the hair was clipped. Data are expressed as the average number of hair follicles produced using the Aderans Hair Patch Assay™ and the error bars represent standard deviation.

FIG. 3 is a bar graph comparing the trichogenicity of dermal cells isolated from skin from live anesthetized mice and sacrificed mice. For each set of mice, dermal cells were isolated from skin from which hair was plucked and from areas of skin immediately adjacent in which the hair was clipped. Data are expressed as the average number of hair follicles produced using the Aderans Hair Patch Assay™ and the error bars represent standard deviation.

### DETAILED DESCRIPTION OF THE INVENTION

### I. Definitions

As used herein, “trichogenicity” or “trichogenic activity” refers to the ability of populations of cells to form new hair follicles when transplanted into skin.

As used herein, the term “effective amount” refers to an amount of dermal and, optionally, other cells such as epidermal cells sufficient to induce hair follicle formation or to induce vellus hair follicles to become terminal hair follicles when introduced into a subject.

As used herein, the term “isolated” is meant to describe cells that are in an environment different from that in which the cells naturally occur e.g., separated from their natural milieu such as by separating dermal cells from a hair follicle.

The terms “subject”, “patient” and “host” are used interchangeably and refer to mammal including a mouse, rat or laboratory animal. A preferred subject is human.

As used herein, the term “skin” refers to the outer covering of an animal. In general, the skin includes the dermis and the epidermis.

As used herein, “dermal cells” or “a population of dermal cells” refers to a population of cells that contains at least 30%, 40%, 50%, 60%, 70%, 80%, 90%, 95%, 97%, 98%, 99% or 100% dermal cells. Methods for identifying a cell as a dermal cell are known in the art.

As used herein, “epidermal cells” or “a population of epidermal cells” refers to a population of cells that contains at least 30%, 40%, 50%, 60%, 70%, 80%, 90%, 95%, 97%, 98%, 99% or 100% epidermal cells. Methods for identifying a cell as an epidermal cell are known in the art.

### II. Methods for Obtaining Populations of Dermal Cells with Increased Trichogenic Ability

Methods for obtaining populations of dermal cells with increased trichogenic ability are disclosed. In one embodiment, the populations of dermal cells with increased trichogenicity include dermal papilla cells. The methods include inducing local trauma to a section of skin tissue from which follicle-forming dermal cells are to be derived. The examples below demonstrate that populations of dermal cells with increased trichogenic activity can be derived from skin tissue that has been subjected to local trauma. The methods further include obtaining the skin tissue that has been subjected to the local trauma after a period of time that is sufficient to allow for increased trichogenicity of dermal cells and dissociating the skin tissue into populations of dermal cells and epidermal cells.

A. Methods for Increasing the Trichogenicity of Populations of Dermal Cells

It has been discovered that the trichogenicity of populations of dermal cells can be increased by inducing local trauma to skin tissue that serves as a source of the dermal cells. The dermal cells with increased trichogenicity may then be isolated for use in applications. Increased trichogenic activity in the population of dermal cells may result from an increased ability of some or all of the dermal cells in the population to form new follicles. Additionally or alternatively, increased trichogenicity of a population of dermal cells may result from an enrichment of dermal cells in the population with the ability to form new follicles. In one embodiment, increased trichogenicity of a population of dermal cells results from an increased ability of some or all of the dermal cells in the population to form new follicles. In another embodiment, increased trichogenicity of a population of dermal cells results from an enrichment of dermal cells in the population with the ability to form new follicles.

In one embodiment, the increased trichogenic activity of dermal cells dissociated from skin tissue that has been locally traumatized results from the induction of a new anagen growth phase of the hair cycle.

Local trauma of the skin can be induced in the skin tissue by a variety of methods, including by mechanical and chemical means. In one embodiment, local trauma is induced in a section of skin tissue by mechanical means. Suitable mechanical means include, but are not limited to, abrasion or scratching of the epidermal surface.

In a preferred embodiment, local trauma of the skin is induced by hair shaft plucking. Hair shaft plucking can be achieved using any suitable means. In one embodiment, hair shaft plucking is achieved using tweezers or epilators. In another embodiment, hair shaft plucking is achieved by waxing. In this method wax is warmed to allow it to be spread easily over the skin in the direction of hair growth. The hair becomes embedded in the wax, as it cools and solidifies. The wax is then pulled off in the opposite direction of the hair growth, pulling the hairs out of the follicles. Suitable waxes include animal waxes, vegetable waxes, mineral waxes, petroleum waxes and synthetic waxes.

Other viscous and/or adhesive materials can be used similarly to wax to adhere to hair shafts for removal from the skin. Resins are hydrocarbon secretions produced by plants. The resin produced by most plants is a viscous liquid, composed mainly of volatile fluid terpenes, with lesser components of dissolved non-volatile solids which make resin viscous and adhesive. A particularly suitable resin is rosin, which is a solid resin obtained from pine trees and other conifers. Highly concentrated sugar solutions may also be used. Waxes, resins and concentrated sugar solutions may also be used in suitable combinations.

In another embodiment, local trauma of the skin is achieved by treatment of the epidermal surface with chemicals or compounds that are caustic or irritating to the skin. Suitable skin irritants include chemicals or compounds known to induce histological and/or immunological responses. Suitable caustic chemicals include mild or strong acids or bases. Exemplary compounds that cause skin irritation include, but are not limited to, retinoic acid and cyclosporine derivatives.

In another embodiment, lasers are used to induce local trauma to the skin. Methods for inducing trauma to the skin using lasers are well known in the art. For example, laser hair removal, laser tattoo removal, laser resurfacing and laser eye surgery in the field of vision correction have become widely practiced cosmetic therapies since the mid 1990s. Combinations of various laser parameters (energy level, wave length, focus spot size, laser pulse width, etc), may be used to induce local trauma to with human skin tissue to achieve specific results. The laser can cause localized damage by selectively heating dark target matter, e.g. melanin, in the area that causes hair growth, (the follicle), while not heating the rest of the skin. For example, when a laser is applied in a laser tattoo removal treatment procedure, the laser works by directing energy toward ink in the skin with highly concentrated colored light beams. These laser beams break up ink particles into tiny fragments which are later cleared up by the body's scavenging cells. The laser selectively targets the pigment of the tattoo without damaging the surrounding skin.

In another embodiment, local trauma to the skin is induced by hyperthermia. Methods for inducing hyperthermia to the skin are well known in the art. For example, local hyperthermia of the skin is commonly induced in the treatment of skin tumors. In this process, skin tissue is exposed to high temperatures (up to 106° F.), to damage and kill cancer cells, or to make cancer cells more sensitive to the effects of radiation and certain anticancer drugs. To induce local hyperthermia of the skin, the skin may be heated externally with high-frequency waves aimed from a device outside the body. To achieve internal heating, one of several types of sterile probes may be used, including thin, heated wires or hollow tubes filled with warm water; implanted microwave antennae; and radio frequency electrodes. Another technique uses ultra-high frequency sound waves to produce heat within the skin. Ultrasound is more easily focused than other energy modalities.

B. Methods for Isolating Populations of Dermal Cells with Increased Trichogenic Ability

Dermal cells are obtained from skin subjected locally to trauma after a period of time sufficient to allow for the dermal cells to possess increased trichogenic activity relative to dermal cells derived from skin that is not subjected to trauma. The examples below demonstrate that dermal cells isolated from traumatized skin have significantly enhanced trichogenic ability as compared to dermal cells obtained from skin that is not traumatized. In one embodiment, trichogenicity of dermal cells is increased after a period of from 30 minutes to 4 weeks following local trauma to the skin. In another embodiment, dermal cells are isolated from locally traumatized skin between 30 minutes and 4 weeks, between 30 minutes and 7 days, between 30 minutes and 3 days, between 30 minutes and 24 hours, between 30 minutes and 180 minutes, between 30 minutes and 120 minutes or between 30 minutes and 60 minutes following induction of the trauma. The examples below demonstrate that trichogenicity of dermal cells is increased by approximately a factor of 6 at 60 minutes after trauma, as compared to dermal cells obtained from non-traumatized skin.

The examples below also demonstrate that the effect of trauma to the skin on increasing the trichogenic ability of dermal cells is localized to the area of skin that is traumatized, and does not extend to skin beyond the area that is directly traumatized. Skin tissue that has been locally traumatized is removed using standard mechanical means. Typically, the section of skin tissue that is removed is approximately 1 cm². Dermal and epidermal cells are aseptically dissociated from the skin tissue by enzymatic and mechanical means. Epidermal cells may also be dissociated from one or more sections of skin from the same donor that were not traumatized, or may be dissociated from skin from a separate donor.

Methods for obtaining dermal and epidermal cells from skin samples are well known in the art. For example, enzymatic and mechanical disruption may be used. Cell strainers containing filters of a particular size may be used to separate the cells based on size.

Dermal and epidermal cell populations that are obtained using these methods are heterogeneous cell populations that include cells of dermal, epidermal and melanocyte origin. Impurities consist of dead cells and debris which do not express the proteins delineating cells into dermal, epidermal or melanocyte origin and a small percentage of cells that are not of a dermal, epidermal, or melanocyte origin.

The cellular content of the cultures may be determined by lineage marker analysis using standard techniques. Molecular markers for dermal and epidermal cells are known in the art. Suitable techniques include flow cytometry to determine cell surface antigen expression and RT-PCR to examine RNA expression.

C. Methods for Determining Trichogenicity of Dermal Cells

Trichogenic activity of populations of dermal cells obtained using the disclosed methods may be assessed using any suitable method known in the art, including, but not limited to the mouse vibrissa organ culture system assay (Jindo, et al., J. Dermatol., 20(12):756-62 (1993)), the Aderans Hair Patch Assay™ (Zheng, et al., J. Invest. Dermatol., 124:867-76 (2005)), the kidney capsule culture assay (Kobayashi and Nishimura, J Invest. Dermatol., 92:278-82 (1989)), or the Lichti/Prouty assay (Lichti, et al., J. Invest. Dermatol., 101:124S-129S (1993)).

In a preferred embodiment, trichogenic activity of populations of dermal cells is determined by the Aderans Hair Patch Assay™. In this assay dissociated dermal and epidermal cells are implanted into the dermis or the subcutis of an immunoincompetent mouse. Using mouse newborn skin cells, new hair follicles typically form in this assay within 8 to 10 days. The newly formed follicle manifests normal hair shafts, mature sebaceous glands, and a natural hair cycle. Although normal cycling hair follicles are formed in this assay, the assay primarily measures the ability of cells or combinations of cells to form new follicles.

### III. Methods for Using Dermal Cells with Increased Trichogenic Ability

Populations of dermal cells with increased trichogenic ability may be used for a number of applications, including implantation into a host to induce the formation of new hair follicles or to induce vellus hair follicles to become terminal hair follicles. In several embodiments, the methods include culturing and expanding dissociated populations of dermal cells with increased trichogenic activity, and implanting the expanded cells into a mammalian host at a site of desired follicle generation.

A. Methods of Culturing and Expanding Dermal Cells with Increased Trichogenic Ability

Populations of dermal cells with increased trichogenic ability obtained using the disclosed methods may be used directly for implantation into a host to induce the formation of new hair follicles or to induce vellus hair follicles to become terminal hair follicles, or they may be cultured and expanded prior to use.

In a preferred embodiment, dermal and epidermal cells are cultured and expanded prior to implantation to obtain a sufficiently large number of cells suitable for implantation into a host to form new hair follicles or to induce vellus hair follicles to become terminal hair follicles. The dissociated cells are cultured in a manner that maintains the increased trichogenic activity of the dermal cells.

Dermal cells may be cultured separately from epidermal cells or may be co-cultured with epidermal cells. Methods for culturing dissociated dermal and epidermal cells are known in the art. Exemplary methods for culturing dermal cells are provided in Roh, et al., Physiol. Genomics, 19:207-17 (2004) and McElwee, et al., Jour. Invest. Dermatol., 121(6):1267-75 (2003).

Suitable cell culture media include commercially available media, such as Dulbecco's Modified Eagle Medium/Nutrient Mixture F-12 (DMEM/F-12), RPMI-1640 and Ham's F10 (Sigma). The medium may be supplemented as appropriate with serum (such as fetal bovine serum, calf serum or horse serum), hormones or other growth factors (such as insulin, epidermal growth factor, Wnt polypeptides, or transferrin), ions (such as sodium, chloride or calcium), buffers (such as HEPES), nucleosides or trace elements.

B. Hair Follicle Induction

Dermal cells with increased trichogenic activity obtained using the disclosed methods may be used to generate new hair follicles in a subject. Subjects to be transplanted with dermal cells with increased trichogenic ability include any subject that has an insufficient amount of hair or an insufficient rate of hair growth. Suitable subjects include those with androgenetic alopecia, alopecia areata, telogen effluvium, thyroid disease, nutritional deficiencies, discoid lupus erythematosus, lichen planus, genetic pattern baldness or with hormonal disorders that decrease hair growth or cause loss of hair. Subjects may have these conditions or be at risk for the development of these conditions, based on genetic, behavioral or environmental predispositions or other factors. Other suitable subjects include those that have received a treatment, such as chemotherapy, or radiation that causes a decrease in hair growth or a loss of hair. Other suitable subjects include subjects that have suffered scalp or hair trauma, have structural hair shaft abnormalities, or that have had a surgical procedure, such as a skin graft, which results in an area of skin in need of hair growth.

Dermal and epidermal cell populations may be implanted into the subject in an area where increased hair growth is desired. Preferred locations for implantation include the subject's scalp, face or eyebrow area.

The cells that are implanted into the subject may be autologous, allogenic or xenogenic. In one embodiment, dermal and epidermal cells are obtained from skin sections from a single allogenic donor or are autologous. In another embodiment, dermal and epidermal cells are obtained from skin sections from more than one donor. For example, dermal cells may be derived from one donor and epidermal cells from another donor. In a preferred embodiment, the cells that are implanted are autologous.

Dermal and epidermal cells are combined at an appropriate ratio prior to implanting into the subject. Suitably, the ratio of epidermal cells to dermal cells is in the range of about 0:1, 1:1, 1:2, or 1:10. Dermal and epidermal cells may be further combined with additional cell types, such as melanocytes, fat cells, pre-adipocytes, endothelial cells, and bone marrow cells prior to implantation. The dermal and epidermal cells to be implanted may be subjected to physical and/or biochemical aggregation prior to implanting to induce and/or maintain aggregation of the cells within the transplantation site. For example, the cells can be aggregated through centrifugation of the culture. Additionally, or alternatively, a suitable aggregation enhancing substance may be added to the cells prior to, or at the time of, implantation. Suitable aggregation enhancing substances include, but are not limited to, glycoproteins such as fibronectin or glycosaminoglycans, dermatan sulfate, chondroitin sulfates, proteoglycans, heparin sulfate and collagen.

The cells may be implanted into a subject using routine methods known in the art. Various routes of administration and various sites can be used. For example, the cells can be introduced directly between the dermis and the epidermis of the outer skin layer at a treatment site. This can be achieved by raising a blister on the skin at the treatment site and introducing the cells into fluid of the blister. The cells may also be introduced into a suitable incision extending through the epidermis down into the dermis. The incision can be made using routine techniques, for example, using a scalpel or hypodermic needle. The incision may be filled with cells generally up to a level in direct proximity to the epidermis at either side of the incision. In a preferred embodiment, the cells are introduced using a delivery device as described in U.S. Published Application No. 2007/0233038.

In another embodiment, a plurality of small closely spaced perforations is formed in the skin into which the cells are transplanted. For example, the plurality can include at least 10, 50, 100, 500, or 1000 perforations. Each perforation can be filled with a large plurality of cells. The size and depth of the perforations can be varied. The lateral extent of individual perforations can be minimized, and limited to approximately 2 mm or 5 mm. The depth of the perforations can be greater than the full depth of the epidermis, for example, extending at least 1 mm or at least 3 mm into the dermis. The perforations in the skin can be fowled by routine techniques and can include the use of a skin-cutting instrument, e.g., a scalpel, a trocar or a hypodermic needle or a laser (e.g., a low power laser). Alternatively, a multiple-perforation apparatus can be used having a plurality of spaced cutting edges formed and arranged for simultaneously forming a plurality of spaced perforations in the skin. The cells can be introduced simultaneously into a plurality of perforations in the skin.

The epidermal and dermal cells may be combined with a pharmacologically suitable carrier such as saline solution or phosphate buffered saline solution. In a preferred embodiment the carrier is a suitable culture medium, such as Dulbecco's Phosphate Buffered Saline (“DPBS”), DMEM, D-MEM-F-12 or HYPOTHERMOSOL-FRS from BioLifeSolutions (Bothell, Wash.). The cells may also be combined with preservation solution such as a solution including, but not limited to, distilled water or deionized water, mixed with potassium lactobionate, potassium phosphate, raffinose, adenosine, allopurinol, pentastarch prostaglandin E1, nitroglycerin, and/or N-acetylcysteine into the solution. Suitably, the preservation solution employed may be similar to standard organ and biological tissue preservation aqueous cold storage solutions such as HYPOTHERMOSOL-FRS from BioLifeSolutions (Bothell, Wash.).

The cells and the carrier may be combined to form a suspension suitable for injection. Each opening is implanted with an effective amount of cells to generate a new hair follicle in that opening. The number of cells introduced into each opening can vary depending on various factors, for example, the size and depth of the opening and the overall viability and trichogenic activity of the cells. The dosage of cells to be injected is typically between about one million to about 4 million cells per square cm. In one embodiment about 50,000 to about 2,000,000 cells are delivered per injection. The cell concentration can be about 5,000 to about 1,000,000 cells/μl, typically about 50,000 cells/μl to about 75,000 cells/μl. A representative volume of cells delivered per injection is about 1 to about 10 μl, preferably about 4 μl. In one embodiment, 1 to 100 injections per cm², typically 1 to 30 injections per cm² are made in the skin, preferably the scalp.

The use of dermal and/or epidermal cells derived from an allogenic source may require administration of an immunosuppressant, alteration of histocompatibility antigens, or use of a barrier device to prevent rejection of the implanted cells. Cells can be administered alone or in conjunction with a barrier or agent for inhibiting or reducing immune responses against the transplanted cells in a recipient subject. For example, an immunosuppressive agent can be administered to a subject to inhibit or interfere with normal response in the subject. The immunosuppressive agent can be an immunosuppressive drug that inhibits T cell/or B cell activity in the subject. Examples of immunosuppressive drugs are commercially available (e.g., cyclosporin A from Sandoz Corp. East Hanover, N.J.). An immunosuppressive agent, e.g., drug, can be administered to a subject at a dosage sufficient to achieve the desired therapeutic effect (e.g., inhibition of rejection of the cells).

The immunosuppressive agent can also be an antibody, an antibody fragment, or an antibody derivative that inhibits T cell activity in the subject. Antibodies capable of depleting or sequestering T cells can be, e.g., polyclonal antisera, e.g., anti-lymphocyte serum; and monoclonal antibodies; e.g., monoclonal antibodies that bind to CD2, CD3, CD4, CD8 or CD40 on the T cell surface. Such antibodies are commercially available, e.g., from American Type Culture Collection, e.g., OKT3 (ATCC CRL 8001). An antibody can be administered for an appropriate time, e.g., at least 7 days, e.g., at least 10 days, e.g., at least 30 days, to inhibit rejection of cultured DP cells following transplantation. Antibodies can be administered intravenously in a pharmaceutically acceptable carrier, e.g., saline solution.

In some embodiments, the subject is treated, topically and/or systematically, with a hair growth promoting substance before, at the same time as, and/or after the transplantation of cells to enhance hair growth. Suitable hair growth promoting substances can include, e.g., minoxidil (available from the Upjohn Co. of Kalamazoo, Mich.), finasteride, cyclosporin, and natural or synthetic steroid hormones and their enhancers and antagonists, e.g., anti-androgens. Following injection, the wound may or may not be covered.

C. Terminal Hair Induction

Another embodiment provides a method for inducing vellus hair to become terminal hair. Vellus hair is the fine, non-pigmented hair (peach fuzz) that covers the body of children and adults. Terminal hair is developed hair, which is generally longer, coarser, thicker and darker than the shorter and finer vellus hair. The growth of vellus hair is not affected by hormones; whereas, the growth of terminal hair is affected by hormones. Vellus hair is also present in male pattern baldness.

In one embodiment, dermal cells with increased trichogenic ability are injected into the skin as described above. The dermal cells are obtained as described above and are typically autologous cells. The cells are injected into or adjacent to vellus hair or vellus hair follicles. Multiple injections of dermal cells may be delivered to an area of skin containing vellus hair to induce as many vellus hair follicles as possible to become terminal hair follicles. It will be appreciated that the number of injections and volume of cells to be injected can be routinely developed by one of skill in the art.

In another embodiment, dermal cells with increased trichogenic activity are injected into skin in an amount effective to induce formation of hair follicles and to induce vellus hair follicles to become terminal hair follicles.

Unless defined otherwise, all technical and scientific terms used herein have the same meanings as commonly understood by one of skill in the art to which the disclosed invention belongs. Publications cited herein and the materials for which they are cited are specifically incorporated by reference.

### EXAMPLES

### Example 1

### Effects of Hair Plucking on the Histology of Mouse Skin

Materials and Methods:

C57BL/6 Mouse the Rosin Wax Pluck

Hair pluck was conducted using the classical method for inducing anagen (Stern and Paus, Physiol. Reviews, 81:449-94 (2001)). An equal mixture of melted rosin and beeswax was painted onto an anesthetized, mature mouse with a full fur coat. The rosin-wax coating was allowed to harden after which it was gently peeled off with the attached hair shafts.

Histology

Tissue sections were prepared using standard hematoxylin and eosin technology (Bancroft and Gamble, Theory and Practice of Histological Techniques. Churchill Livingstone; Edinburgh, pgs. 125-138 (2002)) and the histomorphologies of the plucked and control skin were descriptively assessed.

Results:

Histological changes induced in mouse skin by hair plucking were examined by microscopy using hematoxylin and eosin stained tissue sections. Hematoxylin and eosin stained tissue sections from mice whose hair was clipped, but not plucked, were used as negative controls. Microscopy of skin tissue sections one hour after hair plucking revealed that hair plucking induced a mild increase in cellularity and focal empty follicles as compared to skin tissue sections from prepared from mice with clipped hair.

### Example 2

### Effects of Hair Plucking on Trichogenicity of Mouse Dermal Cells

Materials and Methods:

Isolation of Dermal Cells

Skins were weighed and washed twice in Dulbecco's Phosphate Buffered Saline (DPBS) with 3×PSA and diced with crossed scalpel blades in Petri dish into 1-2 mm² pieces. Skin fragments were divided into Petri dishes with no more than 0.5 gram per dish and incubated in 20 ml of collagenase (2.5 mg/ml) plus dispase (2.5 mg/ml) enzyme mixture at 37° C. for 2 hours, followed by 5 ml of 0.25% Trypsin for 30 minutes. The digestion was neutralized with 5 ml soybean trypsin inhibitor (STI). The solution was filtered through a 100 micron cell strainer after trituration ˜100 times with plastic pipettor. After adding DPBS to make the final volume 5 ml, the cell pellet was collected by centrifugation at 1,400 rpm for 5 minutes. Cells were counted and used in the Aderans Hair Patch Assay™ (1×10⁶ per injection) with neonatal epidermal cells or frozen at 1−5×10⁶ per vial in Medium A with 5% DMSO for later analysis.

Assay of Trichogenicity

Trichogenicity was measured using the Aderans Hair Patch Assay™ as described in Zheng, et al., J. Invest. Dermatol., 124:867-76 (2005)). Briefly, the dermal and epidermal cells to be tested are combined and injected into the dermis or subcutis of an immunocompromised mouse. These studies used nude (nu/nu) mice as a transplant recipient. The implant was left in place for from 8 to 30 days and then the skin was collected and newly formed hair follicles were counted under a dissecting microscope.

Results:

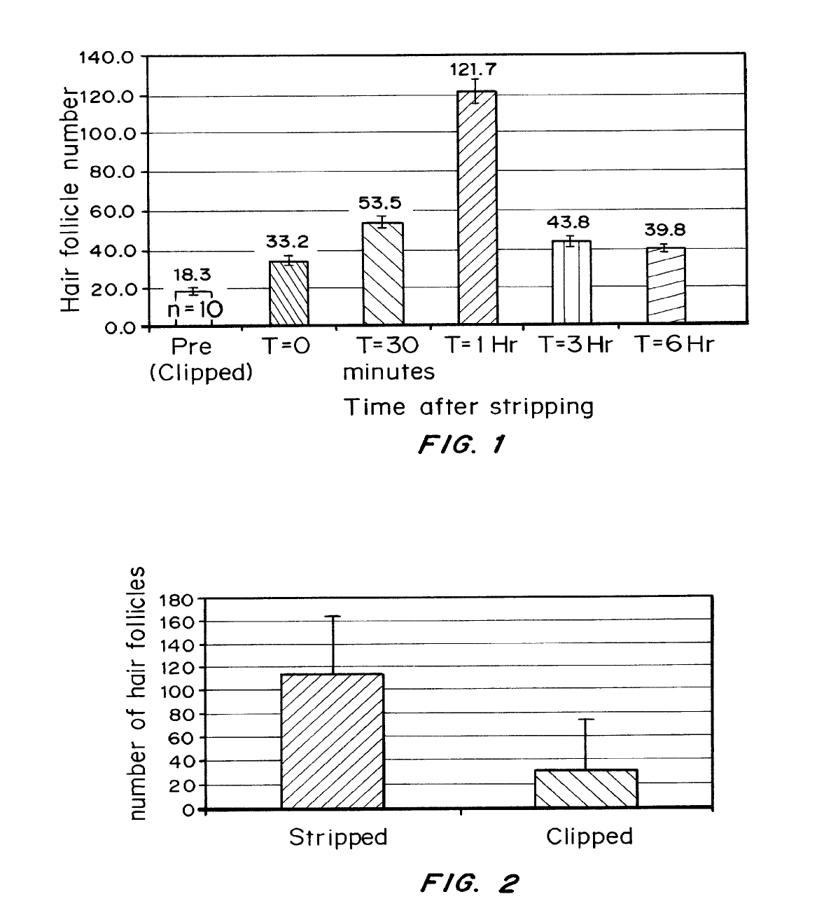

Hair was plucked from mice as described above for Example 1. Dermal cells were then isolated from the plucked skin at various times following hair plucking or from the skin of mice with clipped hair as a negative control. Isolated dermal cells were co-incubated with epidermal cells isolated from the same mice and injected into the dermis or subcutis of nude (nu/nu) immunocompromised mice. This Aderans Hair Patch Assay™ revealed that the trichogenicity of cells isolated from the dermis of mice with plucked hair varies as a function of time after hair plucking (FIG. 1). Trichogenicity was increased most greatly after a period of from 30 to 60 minutes following hair plucking, and was increased by approximately a factor of 6 at 60 minutes after hair plucking, as compared to the non-plucked (hair clipped) control.

### Example 3

### Locality of the Effect of Hair Plucking on Dermal Cell Trichogenicity

Results:

To investigate if the effect of hair plucking on dermal cell trichogenicity is local or spreads to surrounding skin, the hair of the central back area of mice was partially depilated using wax (hair plucking), as described in Example 1 above, and partially clipped. Dermal cells were isolated from the hair plucked areas and from hair clipped areas immediately adjacent to hair plucked areas on the same mouse 60 minutes after hair plucking. Isolated dermal cells were then subjected to the Aderans Hair Patch Assay™, as described above.

Table 1 summarizes the number of hair follicles that developed in the Aderans Hair Patch Assay™ for each set of isolated dermal cells.

TABLE 1
Local effect of hair plucking on trichogenicity of dermal cells.

Number of follicles found in Aderans Hair Patch
Assay ™

| Site | Plucked | Clipped |
|---|---|---|
| Site 1 | 160 | 2 |
| Site 2 | 59 | 5 |
| Site 3 | 200 | 35 |
| Site 4 | 120 | 1 |
| Site 5 | 100 | 5 |
| Site 6 | 110 | 50 |
| Site 7 | 135 | 105 |
| Site 8 | 110 | 20 |
| Site 9 | 130 | 120 |
| Site 10 | 90 | 86 |
| Site 11 | 185 | 18 |
| Site 12 | 130 | 8 |
| Site 13 | 40 | 2 |
| Site 14 | 38 | 3 |
| Average | 114.8 | 32.9 |
| Standard | 48.5 | 41.4 |
| Deviation | | |
| Maximum | 200 | 120 |
| Minimum | 38 | 1 |

The results are summarized graphically in FIG. 2. A paired t-test of the values from Table 1 produced a P-value of 0.00006, indicating that the increase in number of follicles produced by dermal cells from plucked skin as compared to clipped skin is highly statistically significant. The results of these studies indicate that increased trichogenicity of dermal cells achieved by hair plucking is local and does not spread to the surrounding skin.

### Example 4

### Influence of Blood Flow on Increased Trichogenicity Caused by Hair Plucking

Results:

To investigate the effect of active blood flow on increased trichogenicity of dermal cells induced by hair plucking, experiments similar to those described in Example 3 were conducted, using both live anaesthetized mice and sacrificed mice as dermal cell donors. As in Example 3, the hair of the central back area of mice was depilated using wax (hair plucking), or clipped. Dermal cells were isolated from the hair plucked areas and from hair clipped areas immediately adjacent to hair plucked areas on the same mouse 60 minutes after hair plucking or hair clipping. Isolated dermal cells were then subjected to the Aderans Hair Patch Assay™.

Table 2 summarizes the number of hair follicles that developed in the Aderans Hair Patch Assay™ for each set of isolated dermal cells.

TABLE 2
Independence of the pluck effect on blood flow (number of hair
follicles found in the Aderans Hair Patch Assay ™).

| | Live | Live | Dead | Dead | Dead |
|---|---|---|---|---|---|
| Site | Clipped | Plucked | Plucked | Plucked | Clipped |
| Site 1 | 22 | 180 | 49 | 3 | 2 |
| Site 2 | 3 | 44 | 150 | 38 | 0 |
| Site 3 | 4 | 10 | 8 | 0 | 1 |
| Site 4 | 0 | 80 | 15 | 75 | 8 |
| Site 5 | 0 | 12 | 10 | 45 | 0 |
| Site 6 | 0 | 10 | 3 | 13 | 0 |
| Site 7 | 0 | 25 | 3 | 80 | 0 |
| Site 8 | 0 | 18 | 0 | 46 | 0 |
| Site 9 | 15 | 180 | 140 | 140 | 52 |
| Site 10 | 7 | 240 | 220 | 150 | 110 |
| Site 11 | 21 | 170 | 150 | 95 | 57 |
| Site 12 | 60 | 130 | 170 | 170 | 80 |
| Average | 11 | 91.6 | 76.5 | 71.3 | 25.8 |
| Standard | 17.5 | 83.8 | 82.3 | 57.9 | 38.8 |
| Deviation | | | | | |
| Maximum | 60 | 240 | 220 | 170 | 110 |
| Minimum | 0 | 10 | 0 | 0 | 0 |

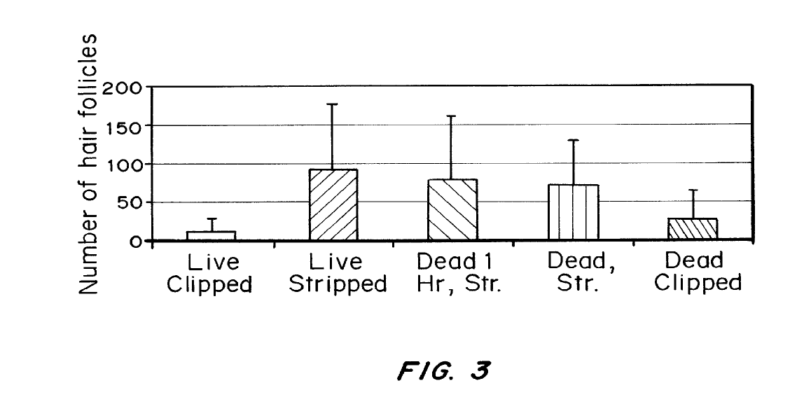

The results are summarized graphically in FIG. 3. The results of these studies demonstrate that increased trichogenicity of dermal cells achieved by hair plucking is similar in live anaesthetized mice and sacrificed mice, indicating that active blood flow is not required for the increase in trichogenicity.

Those skilled in the art will recognize, or be able to ascertain using no more than routine experimentation, many equivalents to the specific embodiments of the invention described herein. Such equivalents are intended to be encompassed by the following claims.

1-12. (canceled)

13. An isolated population of dermal cells obtained by a method comprising,
a) inducing local trauma to a section of skin tissue from which the population of dermal cells is derived;
b) obtaining the area of skin tissue subjected to the local trauma; and
c) dissociating the skin tissue into cells,
wherein the population of dermal cells has an increased trichogenic ability relative to a population of dermal cells dissociated from skin tissue that is not subjected to local trauma.

14-19. (canceled)

20. A system for generating new hair follicles in a mammalian subject comprising:
a) an isolated population of dermal cells according to claim 13; and
b) a means for implanting the dermal cells, and optionally, additional cells into the dermis or subcutis of a mammalian host at a site of desired hair growth.

21. The isolated population of dermal cells of claim 13, wherein the local trauma is induced by mechanical or chemical means.

22. The isolated population of dermal cells of claim 21, wherein the local trauma is induced by hair shaft plucking.

23. The isolated population of dermal cells of claim 21, wherein the local trauma is induced by scratching or abrasion of the skin.

24. The isolated population of dermal cells of claim 21, wherein the local trauma is induced by irritating the skin with lasers, hyperthermia, or caustic chemicals selected from the group consisting of retinoic acid or cyclosporine derivatives.

25. The isolated population of dermal cells of claim 13, wherein the area of skin subjected to the local trauma is obtained after between 30 minutes and 4 weeks, 30 minutes and 180 minutes, 30 minutes and 120 minutes, or 30 minutes and 60 minutes following induction of the local trauma.

26. The isolated population of dermal cells of claim 25, wherein the area of skin subjected to the local trauma is obtained after between 30 and 60 minutes following induction of the local trauma.

27. The isolated population of dermal cells of claim 25, wherein the means for implanting the dermal cells is a scalpel.

28. The isolated population of dermal cells of claim 25, wherein the means for implanting the dermal cells is a trocar or a hypodermic needle.

29. The isolated population of dermal cells of claim 25, wherein the means for implanting the dermal cells is a laser.
